(19) United States
(12) Patent Application Publication
(10) Pub. No.: US 2025/0349397 A1
(43) Pub. Date: Nov. 13, 2025

(54) Recording Medium, Information Processing Method, and Information Processing Device

(71) Applicant: Vision Care Inc., Kobe-shi (JP)

(72) Inventors: Masayo Takahashi, Kobe-shi (JP); Tadao Maeda, Kobe-shi (JP); Takesi Matsuyama Hoyos, Kobe-shi (JP)

(21) Appl. No.: 19/191,387

(22) Filed: Apr. 28, 2025

(30) Foreign Application Priority Data
May 8, 2024 (JP) 2024-076036

(51) Int. Cl. G16H 10/20; G06F 16/3329

(52) U.S. Cl. CPC G16H 10/20; G06F 16/33295

(57) ABSTRACT

Provided are a recording medium, an information processing method, and an information processing device that can reduce workload of medical professionals associated with explanation of informed consent forms.

A computer readable non-transitory recording medium recording an information processing program causes a computer to execute: acquiring a key point sentence related to content of a consent form for a medical procedure or a clinical trial; reading a sentence related to the acquired key point sentence from a database; acquiring an explanatory sentence generated by inputting the key point sentence and the read sentence to a language model; and outputting the acquired explanatory sentence.

### REFERENCE TO RELATED APPLICATIONS

This application claims priority to Japanese Patent Application No. 2024-076036 filed on May 8, 2024. The entire content of the priority application is incorporated herein by reference.

### BACKGROUND

When a medical procedure or research implementation involves interventions including a treatment procedure and collection of a sample, such as blood, on a subject, guidelines and directives stipulate that, from an ethical standpoint, the subjects must understand the content of an informed consent form and give their voluntary consent before the treatment procedure and the like are performed.

Japanese Patent Application Laid-Open No. 2020-52998 discloses a system that can easily modify the content of consent and add information to the content of consent for patient information including an electronic consent form and can guarantee the authenticity of the content of the patient information.

However, even when it is easy to modify the content of consent and to add information to the content of consent, at the site where consent is acquired, consent may be acquired frequently, or the informed consent form may be long, resulting in an increase in the number of items to be explained to the subjects. Further, when the content of the explanation is difficult for the subject to understand, the effort required for the medical professionals to explain the informed consent form is enormous.

### SUMMARY

The present disclosure has been made in view of the above circumstances, and an object of the present invention is to provide an information processing program, an information processing method, and an information processing device that can reduce workload of medical professionals associated with explanation of an informed consent form.

The present application includes a plurality of means for achieving the above object. As an example, there is provided an information processing program causing a computer to execute: acquiring a key point sentence related to content of a consent form for a medical procedure or a clinical trial; reading a sentence related to the acquired key point sentence from a database; acquiring an explanatory sentence generated by inputting the key point sentence and the read sentence to a language model; and outputting the acquired explanatory sentence.

According to the present disclosure, it is possible to reduce the workload of the medical professionals associated with the explanation of the informed consent form.

The above and further objects and features of the invention will more fully be apparent from the following detailed description with accompanying drawings.

### BRIEF DESCRIPTION OF DRAWINGS

FIG. 1 is a diagram showing an example of a configuration of an information processing system according to an embodiment.

FIG. 2 is a view showing an example of an informed consent form.

FIG. 3 is a view showing an example of an FAQ.

FIG. 4 is a view showing an example of a scientific background explanatory document.

FIG. 5 is a view showing an example of a key point sentence in the informed consent form and context information related to the key point sentence.

FIG. 6 is a view showing an example of a prompt for generating an explanatory sentence (informed consent form).

FIG. 7 is a view showing an example of an explanatory sentence generated by a large language model.

FIG. 8 is a view showing a first example of a prompt for an answer to a question sentence.

FIG. 9 is a view showing a second example of the prompt for the answer to the question sentence.

FIG. 10 is a view showing an example of a prompt for generating an answer sentence.

FIG. 11 is a view showing an example of an answer sentence generated by the large language model.

FIG. 12 is a view showing an example of a prompt for controlling or managing a response generated by the large language model.

FIG. 13 is a view showing a first example of a processing procedure performed by an information processing device.

FIG. 14 is a view showing the first example of the processing procedure performed by the information processing device.

FIG. 15 is a view showing a second example of the processing procedure performed by the information processing device.

FIG. 16 is a diagram showing an example of doctor's feedback (consent status) on the informed consent form.

FIG. 17 is a view showing an example of a dialogue log between a user and the large language model.

### DESCRIPTION

An embodiment of the present invention will now be described with reference to the accompanying drawings.

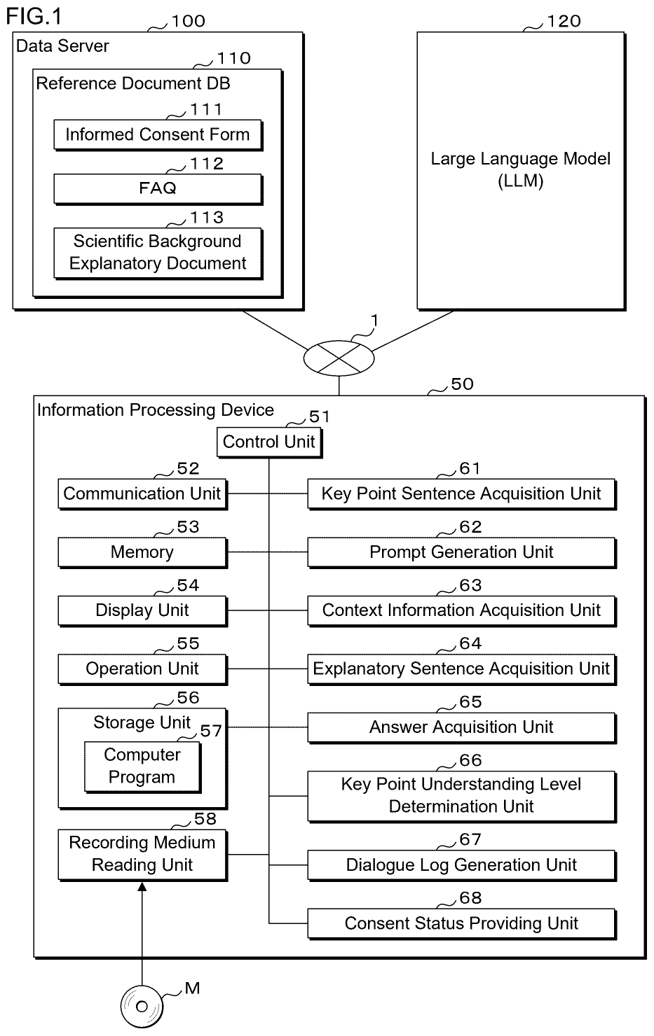

FIG. 1 is a diagram showing an example of a configuration of an information processing system according to this embodiment. The information processing system includes an information processing device 50. The information processing device 50 is connected to a data server 100 and a large language model (LLM) 120 via a communication network 1.

The large language model 120 is a machine learning model that learns language patterns from existing text data and generates or understands text, such as sentences or conversations, using natural language processing technology. The large language model 120 divides text data into small chunks and performs an embedding process on each of the divided chunks to generate a vector representation (embedding) of each chunk. In addition, the embedding process includes a process of quantifying the type of chunk and a process of quantifying a positional relationship between the chunks. Therefore, each chunk is represented as a point in a high-dimensional space, the chunks with similar meanings are disposed at positions close to each other in the high-dimensional space, and the chunks with different meanings are disposed at positions far away from each other.

The large language model 120 can numerically capture the semantic relatedness and similarity between the chunks and can understand the semantics, that is, meaning, of the text data. In addition, the large language model 120 includes an attention mechanism. When linearly combining the numerical representations of each chunk calculated by the embedding process, the large language model 120 appropriately adjusts the magnitude of a linear combination coefficient to obtain a numerical representation expressing a complex structure of a sentence.

The large language model 120 is configured as a deep neural network. For example, models, such as GPT-4, GPT-3.5, BERT, LaMDA, PaLM, and LLaMA, or new models that will be developed and used in the future can be used. The large language model 120 according to this embodiment can provide appropriate responses to questions posed by a user (subject).

The data server 100 includes a reference document DB 110. The reference document DB 110 records an informed consent form 111, an FAQ 112, and a scientific background explanatory document 113. The informed consent form 111 is a document that provides an explanation to a subject in order to obtain the consent of the subject when a medical procedure or research implementation involves interventions including a treatment procedure, collection of a sample, such as blood, and surgery, on the subject. The FAQ 112 is a list of questions frequently asked by the subjects and answers to the questions when the consent of the subjects is obtained. The scientific background explanatory document 113 is a document that explains the scientific background related to the content of the informed consent. The informed consent form 111, the FAQ 112, and the scientific background explanatory document 113 will be described in detail below.

The information processing device 50 includes a control unit 51 that controls the entire device, a communication unit 52, a memory 53, a display unit 54, an operation unit 55, a storage unit 56, a recording medium reading unit 58, a key point sentence acquisition unit 61, a prompt generation unit 62, a context information acquisition unit 63, an explanatory sentence acquisition unit 64, an answer acquisition unit 65, a key point understanding level determination unit 66, a dialogue log generation unit 67, and a consent status providing unit 68.

The control unit 51 may be configured by incorporating a required number of central processing units (CPUs), micro-processing units (VPUs), graphics processing units (GPUs), and the like. Further, the control unit 51 may be configured by combining digital signal processors (DSPs), field-programmable gate arrays (FPGAs), and the like.

The communication unit 52 includes a communication module and has a function of communicating with the data server 100 and the large language model 120 via the communication network 1.

The display unit 54 is configured as a liquid crystal display, an organic EL display, or the like and displays required information to provide a user interface (UI) for the user. The display unit 54 includes a microphone and a speaker, can input voice, and can output voice.

The operation unit 55 is configured, for example, as a touch panel and can be used to operate icons displayed on the display unit 54, to move and operate a cursor, to input letters, and the like. The operation unit 55 may be configured by buttons, switches, and the like or may be configured by a keyboard, a mouse, and the like. The operation unit 55 receives the operation of the user and provides a UI for the user.

The storage unit 56 can be configured as a semiconductor memory or a hard disk and stores a computer program 57 (program product) and required information.

The computer program 57 recorded on a recording medium (for example, an optically readable disk storage medium such as a CD-ROM) M can be read by the recording medium reading unit 58 and stored in the storage unit 56. In addition, the computer program 57 may be downloaded from an external device via the communication unit 52 and stored in the storage unit 56.

The memory 53 can be configured as a semiconductor memory such as a static random access memory (SRAM), a dynamic random access memory (DRAM), or a flash memory. The computer program 57 can be deployed in the memory 53, and the control unit 51 can execute the computer program 57. The control unit 51 can execute processes defined by the computer program 57. That is, the processes executed by the control unit 51 are also processes executed by the computer program 57. In addition, the processes of each of the key point sentence acquisition unit 61, the prompt generation unit 62, the context information acquisition unit 63, the explanatory sentence acquisition unit 64, the answer acquisition unit 65, the key point understanding level determination unit 66, the dialogue log generation unit 67, and the consent status providing unit 68 can be implemented by the processes of the computer program 57. That is, the control unit 51 can execute each of the processes of the key point sentence acquisition unit 61, the prompt generation unit 62, the context information acquisition unit 63, the explanatory sentence acquisition unit 64, the answer acquisition unit 65, the key point understanding level determination unit 66, the dialogue log generation unit 67, and the consent status providing unit 68.

The key point sentence acquisition unit 61 acquires a key point sentence related to the content of a consent form for a medical procedure or a clinical test from the informed consent form 111. Specifically, the key point sentence acquisition unit 61 acquires the key point sentence in the informed consent form 111. The key point sentence is a sentence written in each item of the informed consent form and is equivalent to the summary or title of each item of the informed consent form.

The context information acquisition unit 63 searches the reference document DB 110 to acquire context information which is a sentence related to the key point sentence acquired by the key point sentence acquisition unit 61. In addition, when the user asks a question of the large language model 120, the context information acquisition unit 63 can acquire the question and search the reference document DB 110 to acquire context information which is a sentence related to the question.

The prompt generation unit 62 generates a prompt which is an input to the large language model 120. The prompt includes elements including a task (command) desired to be executed by the large language model 120, information required for the large language model 120 to understand the task and to make an appropriate determination (context information, background information, and the like), data, such as a question to the large language model 120, and an output format of an answer to the question.

The prompt generation unit 62 can generate the prompt based on the key point sentence acquired by the key point sentence acquisition unit 61 and the context information acquired by the context information acquisition unit 63. Furthermore, the prompt generation unit 62 can generate the prompt based on the user's question and context information acquired by the context information acquisition unit 63. The prompt generated by the prompt generation unit 62 can be displayed on the display unit 54.

The prompt generation unit 62 can input the generated prompt to the large language model 120. In this case, the text data included in the prompt is divided into chunks using, for example, LangChain which is one of libraries, and each of the divided chunks is converted into a vector representation.

When the prompt generated based on the key point sentence and the context information is input to the large language model 120, the large language model 120 generates an informed consent form (explanatory sentence) and outputs the generated informed consent form. The explanatory sentence acquisition unit 64 acquires the informed consent form generated by the large language model 120. The informed consent form acquired by the explanatory sentence acquisition unit 64 can be displayed on the display unit 54.

When the prompt generated based on the user's question and the context information is input to the large language model 120, the large language model 120 generates an answer sentence to the question and outputs the generated answer sentence. The answer acquisition unit 65 acquires the answer sentence generated by the large language model 120. The answer sentence acquired by the answer acquisition unit 65 can be displayed on the display unit 54.

The key point understanding level determination unit 66 determines whether the user has understood the sentence related to the key point sentence for each key point, based on the informed consent form output by the large language model 120 and the user's feedback on the answer sentence (for example, the user's answer indicating the level of understanding, whether or not there is a question, the content of the question, and the like). The level of understanding can be classified, for example, into (1) “understood” and (2) “do not understand very well”, and “do not understand very well” can be classified, for example, into (1) “difficult to understand” or (2) “There is a concern”. Further, these classifications are only examples, and the present disclosure is not limited thereto.

The dialogue log generation unit 67 can generate and record a log of the dialogue between the user and the large language model 120.

The consent status providing unit 68 can generate a consent status, such as whether or not the user has agreed to the informed consent form or whether or not the explanation of the informed consent by a doctor is required, based on the level of understanding determined by the key point understanding level determination unit 66, and output the generated consent status. That is, the consent status providing unit 68 can collect points that the user does not understand, points that are difficult for the user to understand, or points that the user is worried or concerned about as the consent status for the informed consent form and can provide the collected consent status as feedback on medical professionals such as doctors.

Next, the reference document DB 110 will be described.

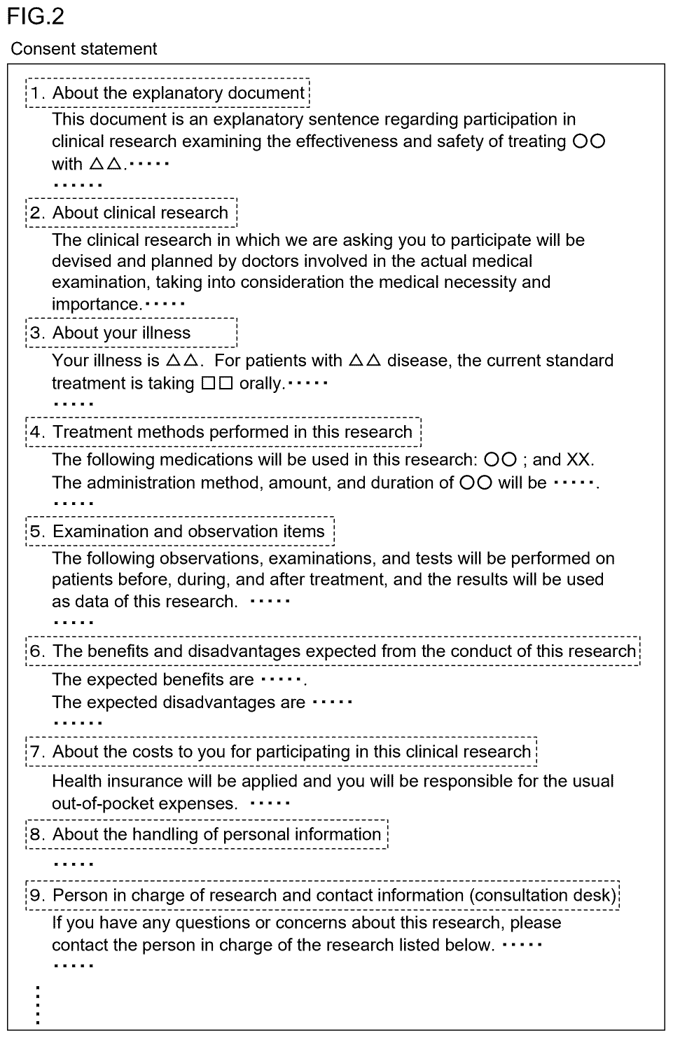

FIG. 2 is a view showing an example of the informed consent form 111. The informed consent form 111 is a document that explains to the user (subject) in order to obtain the consent of the subject when a medical procedure or research implementation involves interventions including a medical procedure, collection of a sample, such as blood, and surgery, on the subject. The informed consent form 111 includes key point sentences equivalent to the summary or title of the content of the explanation and explanatory documents related to the key point sentences. In the example shown in FIG. 2, each item (for convenience, represented by numbers 1, 2, . . . ) is composed of a key point sentence surrounded by a dashed line and an explanatory document related to the key point sentence. For example, a key point sentence for a first item is “About the explanatory document”, and an explanatory document states that “This document is an explanatory sentence regarding participation in clinical research examining the effectiveness and safety of treating ◯◯ with ΔΔ . . . .”.

A key point sentence for a second item is “About clinical research”, and an explanatory document states that “The clinical research in which we are asking you to participate will be devised and planned by doctors involved in the actual medical examination, taking into consideration the medical necessity and importance . . . . ”.

A key point sentence for a third item is “About your illness”, and an explanatory document states that “Your illness is ΔΔ. For patients with ΔA disease, the current standard treatment is taking □□ orally . . . . ”.

A key point sentence for a fourth item is “Treatment methods performed in this research”, and an explanatory document states that “The following medications will be used in this research: ◯◯; and XX. The administration method, amount, and duration of ◯◯ will be . . . . . . .”.

A key point sentence for a fifth item is “Examination and observation items”, and an explanatory document states that “The following observations, examinations, and tests will be performed on patients before, during, and after treatment, and the results will be used as data of this research . . . . ”.

A key point sentence for a sixth item is “The benefits and disadvantages expected from the conduct of this research”, and an explanatory document states that “The expected benefits are . . . . The expected disadvantages are . . . ”.

A key point sentence for a seventh item is “About the costs to you for participating in this clinical research”, and an explanatory document states that “Health insurance will be applied and you will be responsible for the usual out-of-pocket expenses . . . . ”.

A key point sentence for an eighth item is “About the handling of personal information”. In addition, an explanatory document is omitted.

A key point sentence for a ninth item is “Person in charge of research and contact information (consultation desk)”, and an explanatory document states that “If you have any questions or concerns about this research, please contact the person in charge of the research listed below . . . . ”. The same applies to other key point sentences. Further, the informed consent form shown in FIG. 2 is only an example, and the present disclosure is not limited to the example shown in FIG. 2. The content of the informed consent form will vary depending on the content of the clinical research and the content of the medical procedure on the subject. In addition, the informed consent form 111, the FAQ 112, and the scientific background explanatory document 113 are not single documents but collections of a plurality of documents.

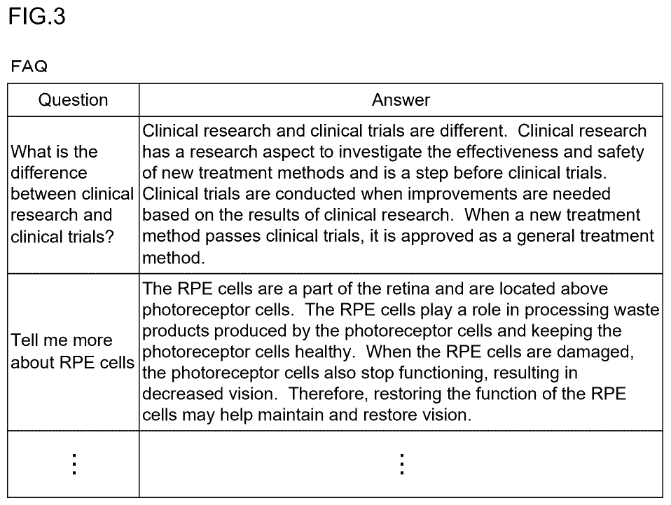

FIG. 3 is a view showing an example of the FAQ 112. The FAQ 112 is a compilation of the questions frequently asked by the subjects when the consent of the subjects is acquired and the answers to the questions. As shown in FIG. 3, the answer to the question “What is the difference between clinical research and clinical trials?” is “Clinical research and clinical trials are different. Clinical research has a research aspect to investigate the effectiveness and safety of new treatment methods and is a step before clinical trials. Clinical trials are conducted when improvements are needed based on the results of clinical research. When a new treatment method passes clinical trials, it is approved as a general treatment method.”

In addition, the answer to the question “Tell me more about RPE cells” is “The RPE cells are a part of the retina and are located above photoreceptor cells. The RPE cells play a role in processing waste products produced by the photoreceptor cells and keeping the photoreceptor cells healthy. When the RPE cells are damaged, the photoreceptor cells also stop functioning, resulting in decreased vision. Therefore, restoring the function of the RPE cells may help maintain and restore vision.” In addition, the FAQs shown in FIG. 3 are only an example, and the present disclosure is not limited to the example shown in FIG. 3. The content of the FAQs will vary depending on the content of the clinical research and the content of the medical procedure on the subject.

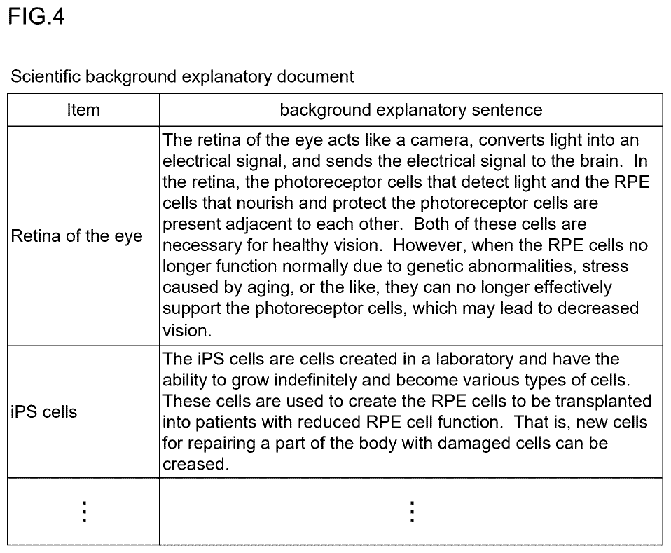

FIG. 4 is a view showing an example of the scientific background explanatory document 113. The scientific background explanatory document 113 is a document that explains the scientific background related to the content of the informed consent. As shown in FIG. 4, items include “Retina of the eye”, “iPS cells”, and the like, and a background explanatory sentence is associated with each item. For example, a background explanatory sentence for the item “Retina of the eye” states that “The retina of the eye acts like a camera, converts light into an electrical signal, and sends the electrical signal to the brain. In the retina, the photoreceptor cells that detect light and the RPE cells that nourish and protect the photoreceptor cells are present adjacent to each other. Both of these cells are necessary for healthy vision. However, when the RPE cells no longer function normally due to genetic abnormalities, stress caused by aging, or the like, they can no longer effectively support the photoreceptor cells, which may lead to decreased vision.”

In addition, a background explanatory sentence for the item “iPS cells” states that “The iPS cells are cells created in a laboratory and have the ability to grow indefinitely and become various types of cells. These cells are used to create the RPE cells to be transplanted into patients with reduced RPE cell function. That is, new cells for repairing a part of the body with damaged cells can be creased.” Further, the scientific background explanatory document shown in FIG. 4 is only an example, and the present disclosure is not limited to the example shown in FIG. 4. The content of the scientific background explanatory document will vary depending on the content of the clinical research and the content of the medical procedure on the subject.

The informed consent form 111, the FAQ 112, and the scientific background explanatory document 113 recorded in the reference document DB 110 are subjected to an embedding process at a required timing and are embedded in the large language model 120. The embedding process is performed by dividing the text data of the informed consent form 111, the FAQ 112, and the scientific background explanatory document 113 into chunks and converting each of the divided chunks into a vector representation. Therefore, the large language model 120 can understand the meaning contained in the informed consent form 111, the FAQs 112, and the scientific background explanatory document 113. The required timing may be the timing when the user uses the large language model 120 or the timing when the reference document DB 110 is updated.

Next, the process of the information processing device 50 (control unit 51) on the large language model 120 and the response of the large language model 120 will be described.

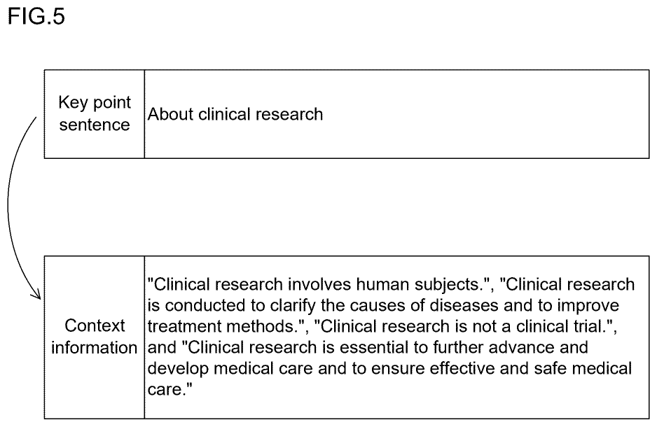

FIG. 5 is a view showing an example of a key point sentence in the informed consent form and context information related to the key point sentence. The control unit 51 (key point sentence acquisition unit 61) acquires the key point sentences from the informed consent form 111. A plurality of key point sentences are prepared in association with a presentation order (for example, the numerical order shown in FIG. 2), and the control unit 51 acquires the key point sentence in the presentation order. The control unit 51 can output an explanatory sentence corresponding to the acquired key point sentence.

Then, the control unit 51 (context information acquisition unit 63) reads a sentence related to the acquired key point sentence from the reference document DB (database) 110 to acquire context information. In this case, the control unit 51 can read a sentence related to the key point sentence from the reference document DB 110 based on features (for example, vector expressions) calculated from the key point sentence and features (vector expressions) associated with the sentence.

In the example shown in FIG. 5, contextual information, such as “Clinical research involves human subjects.”, “Clinical research is conducted to clarify the causes of diseases and to improve treatment methods.”, “Clinical research is not a clinical trial.”, and “Clinical research is essential to further advance and develop medical care and to ensure effective and safe medical care.”, is read as a document related or similar to the vector representation of the key point sentence “About clinical research”. In addition, the context information is only an example, and the present invention is not limited to the example shown in FIG. 5.

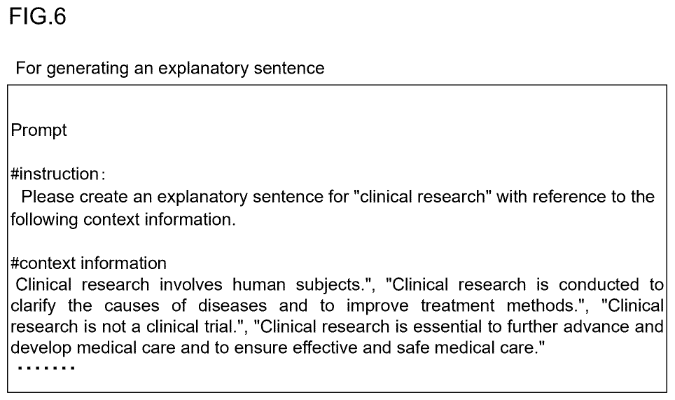

FIG. 6 is a view showing an example of a prompt for generating an explanatory sentence (informed consent form). The control unit 51 (prompt generation unit 62) generates a prompt to be input to the large language model 120 based on the key point sentence and the read context information. In the example shown in FIG. 6, an instruction for the large language model 120 is “Please create an explanatory sentence for “clinical research” with reference to the following context information.”, and the context information is “Clinical research involves human subjects.”, “Clinical research is conducted to clarify the causes of diseases and to improve treatment methods.”, “Clinical research is not a clinical trial.”, “Clinical research is essential to further advance and develop medical care and to ensure effective and safe medical care.”, and the like.

When the prompt shown in FIG. 6 is input, the large language model 120 generates an explanatory sentence (informed consent form) and outputs the generated explanatory sentence to the information processing device 50. The control unit 51 (explanatory sentence acquisition unit 64) acquires the explanatory sentence generated by the large language model 120. The acquired explanatory sentence is displayed on the display unit 54. A plurality of key point sentences are prepared in association with the presentation order, and the large language model 120 can generate explanatory sentences corresponding to the key point sentences in the presentation order and output the explanatory sentences.

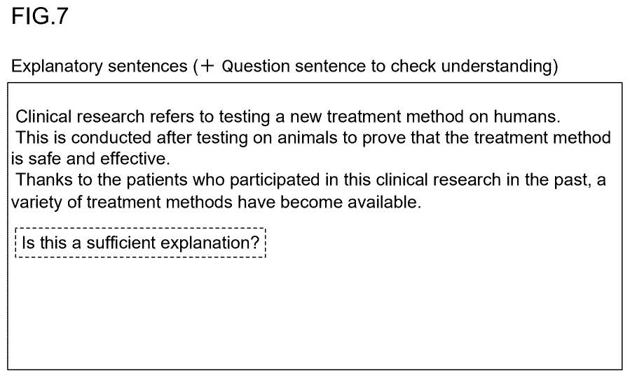

FIG. 7 is a view showing an example of the explanatory sentences generated by the large language model 120. As shown in FIG. 7, an explanatory sentence for the key point sentence “About clinical research” is “Clinical research refers to testing a new treatment method on humans. This is conducted after testing on animals to prove that the treatment method is safe and effective. Thanks to the patients who participated in this clinical research in the past, a variety of treatment methods have become available.”

Further, as shown in FIG. 7, the control unit 51 can output a question sentence to check understanding along with the explanatory sentence. In the example shown in FIG. 7, a question sentence “Is this a sufficient explanation?” is output. In addition, the question sentence is not limited to the example shown in FIG. 7 and may be any sentences that can determine the level of understanding of the user. For example, the question sentence may be “Is this explanation sufficient?”, “Do you have any other questions?”, or “Did you understand this explanation?”. The control unit 51 (answer acquisition unit 65) acquires an answer to the question sentence.

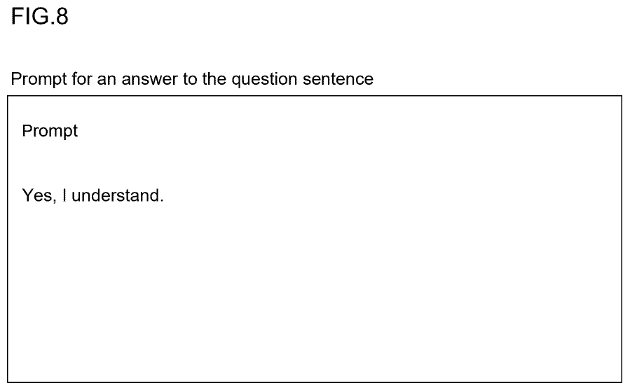

FIG. 8 is a view showing a first example of a prompt for an answer to the question sentence. In the example shown in FIG. 8, the user responds to the explanatory sentence (+the question sentence asking for understanding) shown in FIG. 7 by saying “Yes, I understand.” The control unit 51 (key point understanding level determination unit 66) determines the level of understanding of the user for the key point sentence based on the user's answer. In the example shown in FIG. 8, control unit 51 can determine that the user has understood the key point sentence.

When the control unit 51 determines that the user (explainee) has understood the content of the explanation related to the key point sentence based on the acquired answer, it can output an explanatory sentence corresponding to the next key point sentence in order.

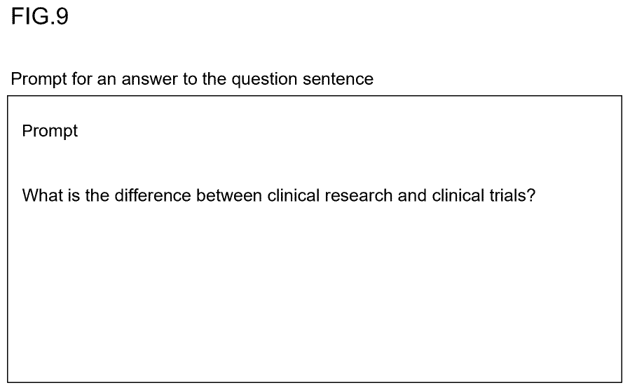

FIG. 9 is a view showing a second example of the prompt for the answer to the question sentence. In the example shown in FIG. 9, the user asks a further question “What is the difference between clinical research and clinical trials?” in response to the explanatory sentence (+the question sentence asking for understanding) shown in FIG. 7. In this case, since the user has not explicitly answered that the user understands as shown in FIG. 8 and has further asked a question, the control unit 51 can determine that the user does not yet fully understand.

When the control unit 51 determines that the user does not understand the content of the explanation related to the key point sentence and that the question sentence has been acquired based on the acquired answer, the control unit 51 reads a sentence related to the question sentence from the reference document DB 110 to acquire the context information. In this case, the control unit 51 can read a sentence related to the question sentence from the reference document DB 110 based on the features (for, vector expressions) obtained from the question sentence and the features (vector expressions) associated with the sentence.

The control unit 51 (prompt generation unit 62) generates the prompt to be input to the large language model 120 based on the question sentence and the read context information.

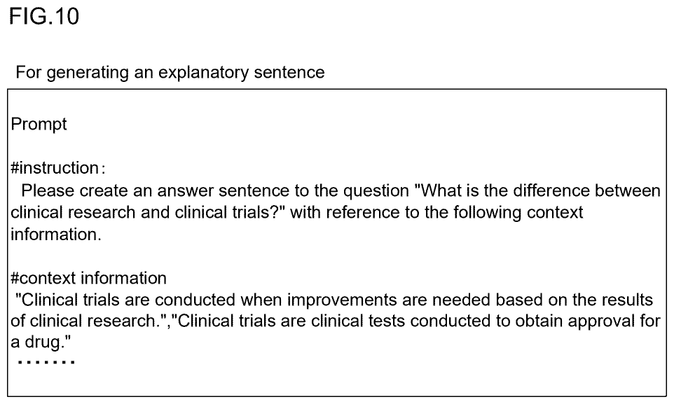

FIG. 10 is a view showing an example of a prompt for generating an answer sentence. In the example shown in FIG. 10, an instruction for the large language model 120 is “Please create an answer sentence to the question “What is the difference between clinical research and clinical trials?” with reference to the following context information.”, and the context information is, for example, “Clinical trials are conducted when improvements are needed based on the results of clinical research.” or “Clinical trials are clinical tests conducted to obtain approval for a drug.”

The control unit 51 inputs the generated prompt to the large language model 120. The large language model 120 generates an answer sentence as a response to the input prompt and outputs the generated answer sentence to the information processing device 50. The control unit 51 (answer acquisition unit 65) acquires the answer sentence generated by the large language model 120. The acquired answer sentence is displayed on the display unit 54.

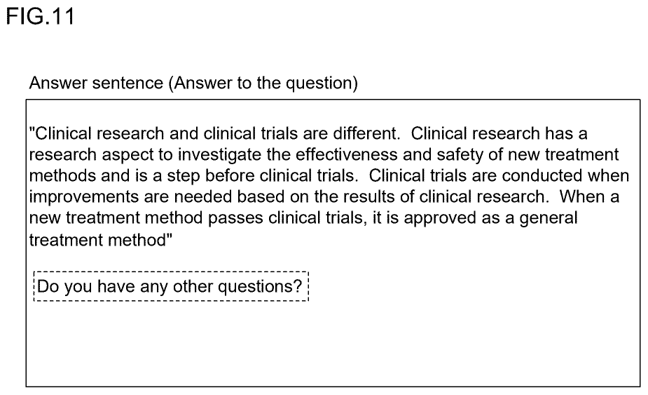

FIG. 11 is a view showing an example of the answer sentence generated by the large language model 120. As shown in FIG. 11, the answer to the question “What is the difference between clinical research and clinical trials?” is “Clinical research and clinical trials are different. Clinical research has a research aspect to investigate the effectiveness and safety of new treatment methods and is a step before clinical trials. Clinical trials are conducted when improvements are needed based on the results of clinical research. When a new treatment method passes clinical trials, it is approved as a general treatment method”.

Further, as shown in FIG. 11, the control unit 51 can output a question sentence to check understanding along with the answer sentence. In the example shown in FIG. 11, a question sentence “Do you have any other questions?” is output. In addition, the question sentence is not limited to the example shown in FIG. 11 and may be any sentences that can determine the level of understanding of the user.

The control unit 51 acquires the user's answer to the answer sentence. When determining that the user understands the answer sentence based on the acquired answer, the control unit 51 can output an explanatory sentence corresponding to the next key point sentence in the presentation order.

The control unit 51 (prompt generation unit 62) can generate a prompt for controlling or managing responses such as the explanatory sentence and the answer sentence generated by the large language model 120.

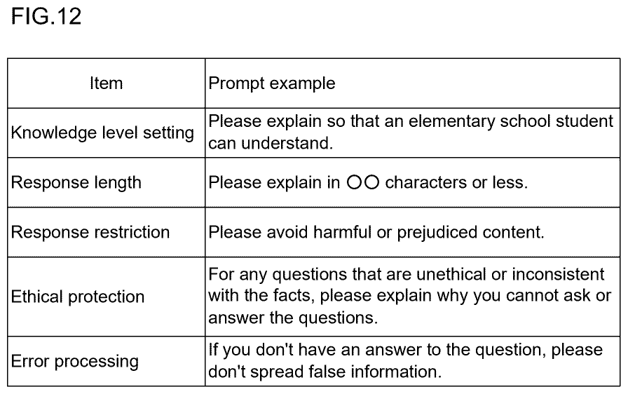

FIG. 12 is a view showing an example of the prompt for controlling or managing the responses generated by the large language model 120. As shown in FIG. 12, items for controlling or managing the responses generated by the large language model 120 include, for example, “knowledge level setting”, “response length”, “response restriction”, “ethical protection”, and “error processing”.

The “knowledge level setting” is for generating responses corresponding to the knowledge level of the user. For example, a prompt “Please explain so that an elementary school student can understand” can be generated and input to the large language model 120. The prompt is not limited to the example shown in FIG. 12 and may be, for example, a prompt “Please explain so that a junior high school student can understand.” or a prompt “Please explain so that a child can understand.”

The “response length” is for adjusting the length such that the user easily accepts the response to deepen the user's understanding while interacting with the large language model 120. For example, a prompt “Please explain in ◯◯ characters or less” can be generated and input to the large language model 120.

The “response restriction” is intended to ensure that the responses generated by the large language model 120 are safe and socially positive and do not contain harmful or prejudiced content. For example, a prompt “Please avoid harmful or prejudiced content.” can be generated and input to the large language model 120.

The “Ethical protection” is intended to ensure that the large language model 120 does not generate incorrect responses when the user's question is unethical or inconsistent with the facts. For example, a prompt “For any questions that are unethical or inconsistent with the facts, please explain why you cannot ask or answer the questions.” can be generated and input to the large language model 120.

“Error processing” is intended to prevent the large language model 120 from spreading false information when the large language model 120 does not know the answer. For example, a prompt “If you don't have an answer to the question, please don't spread false information.” can be generated and input into the large language model 120.

As described above, when a question sentence is unethical or inconsistent with the facts, the control unit 51 can output a sentence pointing out that the question is unethical or inconsistent with the facts and a sentence explaining that the question cannot be answered.

Further, the control unit 51 can give, to the large language model 120, an instruction indicating that the informed consent form does not contain false information and unethical and harmful content. Furthermore, the control unit 51 can give, to the large language model 120, an instruction indicating that the answer sentence does not contain false information and unethical and harmful content.

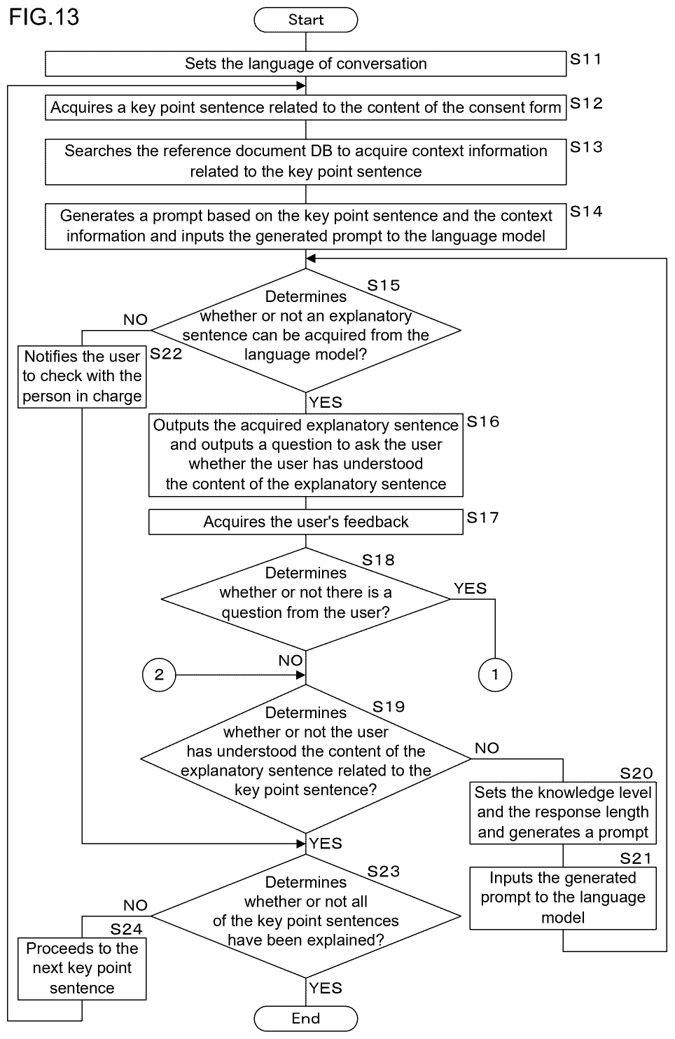

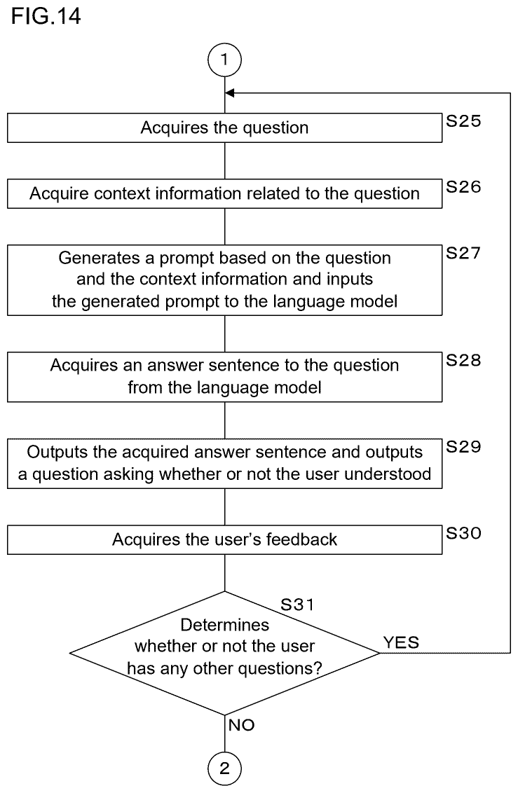

FIGS. 13 and 14 are views showing a first example of a processing procedure performed by the information processing device 50. The processes shown in FIGS. 13 and 14 include a process for outputting the informed consent form. The control unit 51 sets the language of conversation (S11). For the language setting, when the language used by the user can be specified, the specified language is set. When the language used by the user cannot be specified, a default language may be set.

The control unit 51 acquires a key point sentence related to the content of the consent form (S12). Specifically, the control unit 51 can acquire a key point sentence (shown in FIG. 2) from the informed consent form 111. The control unit 51 searches the reference document DB 110 to acquire context information (shown in FIG. 5) related to the key point sentence (S13).

The control unit 51 generates a prompt based on the key point sentence and the context information and inputs the generated prompt (shown in FIG. 6) to the language model (large language model 120) (S14). The control unit 51 determines whether or not an explanatory sentence can be acquired from the language model (S15). When the explanatory sentence can be acquired (YES in S15), the control unit 51 outputs the acquired explanatory sentence and outputs a question to ask the user whether the user has understood the content of the explanatory sentence (S16). The explanatory sentence and question to be output are shown in FIG. 7 as an example.

The control unit 51 acquires the user's feedback (S17). The user's feedback includes the user's answer to the question (shown in FIGS. 8 and 9). The control unit 51 determines whether or not there is a question from the user (S18). When there is no question (NO in S18), the control unit 51 determines whether or not the user has understood the content of the explanatory sentence related to the key point sentence (S19).

When the user does not understand the content of the explanatory sentence related to the key point sentence (NO in S19), the control unit 51 sets the knowledge level and the response length and generates a prompt (S20). The control unit 51 inputs the generated prompt to the language model (S21) and continues the processes in Step S15 and the subsequent steps.

When the explanatory sentence cannot be acquired from the language model (NO in S15), the control unit 51 notifies the user to check with the person in charge (S22) and performs a process in Step S23 which will be described below. When the user understands the content of the explanatory sentence related to the key point sentence (YES in S19), the control unit 51 determines whether or not all of the key point sentences have been explained (S23). When all of the key point sentences have not been explained (NO in S23), the control unit 51 proceeds to the next key point sentence (S24) and continues the processes in Step S12 and the subsequent steps. When all of the key point sentences have been explained (YES in S23), the control unit 51 ends the process.

When the user has a question (YES in S18), the control unit 51 acquires the question (S25) and searches the reference document DB 110 to acquire context information (shown in FIG. 10) related to the question (S26). The control unit 51 generates a prompt based on the question and the context information and inputs the generated prompt (shown in FIG. 10) to the language model (S27).

The control unit 51 acquires an answer sentence (shown in FIG. 11) to the question from the language model (S28). The control unit 51 outputs the acquired answer sentence and outputs a question asking whether or not the user understood (S29). The output answer sentence and question are shown in FIG. 11.

The control unit 51 acquires the user's feedback (S30) and determines whether or not the user has any other questions (S31). When the user has any other questions (YES in S31), the control unit 51 continues the processes in Step S25 and the subsequent steps. When the user has no other questions (YES in S31), the control unit 51 continues the processes in Step S19 and the subsequent steps.

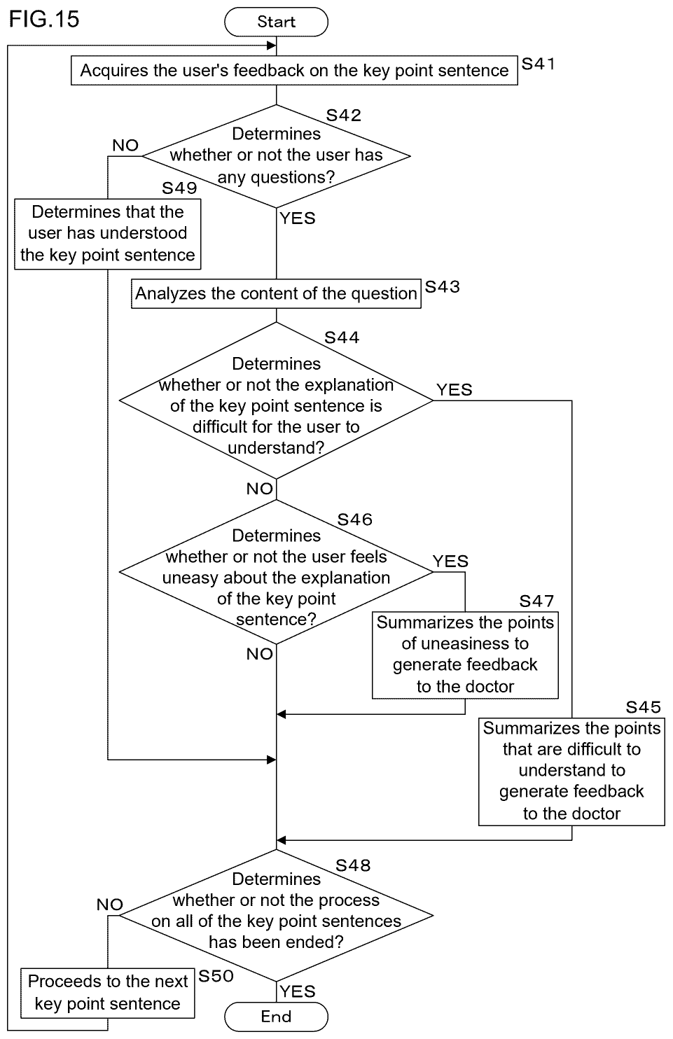

FIG. 15 is a view showing a second example of the processing procedure performed by the information processing device 50. The processes shown in FIG. 15 includes a process for providing the user's consent status which is feedback to the doctor. The control unit 51 acquires the user's feedback on the key point sentence (S41) and determines whether or not the user has any questions (S42). When the user has a question (YES in S42), the control unit 51 analyzes the content of the question (S43) and determines whether or not the explanation of the key point sentence is difficult for the user to understand (S44).

When the explanation of the key point sentence is difficult for the user to understand (YES in S44), the control unit 51 summarizes the points that are difficult to understand to generate feedback to the doctor (S45) and performs a process in Step S48 which will be described below. When the explanation of the key point sentence is easy for the user to understand (NO in S44), the control unit 51 determines whether or not the user feels uneasy about the explanation of the key point sentence (S46).

When the user feels uneasy about the explanation of the key point sentence (YES in S46), the control unit 51 summarizes the points of uneasiness to generate feedback to the doctor (S47) and determines whether or not the process on all of the key point sentences has been ended (S48). When the user does not feel uneasy about the explanation of the key point sentence (NO in S46), the control unit 51 performs the process in Step S48. When the user has no question (NO in S42), the control unit 51 determines that the user has understood the key point sentence (S49) and performs the process in Step S48.

When the process on all of the key point sentences has not been ended (NO in S48), the control unit 51 proceeds to the next key point sentence (S50) and continues the processes in Step S41 and the subsequent steps. When the process on all of the key point sentences has been ended (YES in S48), the control unit 51 ends the process.

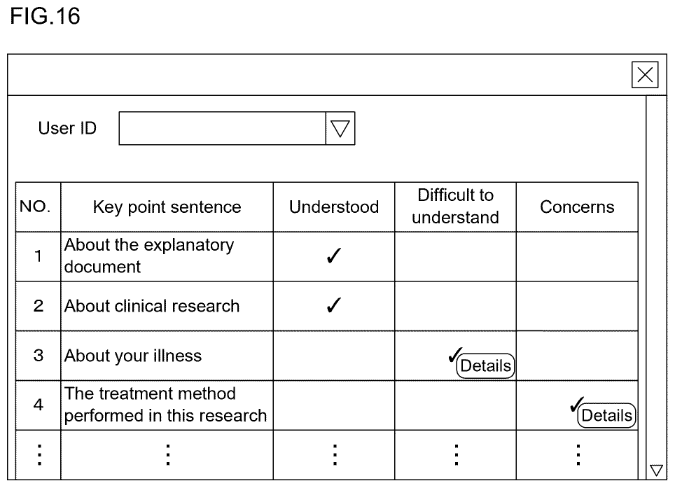

FIG. 16 is a view showing an example of the doctor's feedback (consent status) on the informed consent form. The feedback shown in FIG. 16 can be displayed on a display unit of a terminal device (not shown) used by the doctor or the medical professional. The feedback includes a user ID (for example, a subject number or a patient number), each key point sentence in the informed consent form, and the level of understanding of the user (for example, classified into “understood”, “difficult to understand”, and “concerns”) for the content of each explanatory sentence related to each key point sentence. In addition, a “details” icon is displayed in each of the classifications of “difficult to understand” and “concerns”. That is, the feedback is a consent status obtained by collecting the points that the user does not understand, the points that are difficult for the user to understand, or the points that the user is worried or concerned about.

As shown in FIG. 16, for the key point sentences “About the explanatory document” and “About clinical research”, a check mark is displayed in “understood”. Therefore, the doctor can determine that the user has understood the content of the informed consent form related to the key point sentences “About the explanatory document” and “About clinical research”.

In addition, for the key point sentence “About your illness”, a check mark is displayed in “difficult to understand”. Therefore, the doctor can determine that the user does not fully understand the content of the informed consent form related to the key point sentence “About your illness”. Further, for the key sentence “The treatment method performed in this research”, a check mark is displayed in “concerns”. Therefore, the doctor can determine that the user has concerns about the content of the informed consent form related to the key point sentence “The treatment method performed in this research”.

The doctor can easily determine which key point sentence the user understands, which key point sentence the user does not understand, or which key point sentence the user has concerns about, with reference to the feedback shown in FIG. 16.

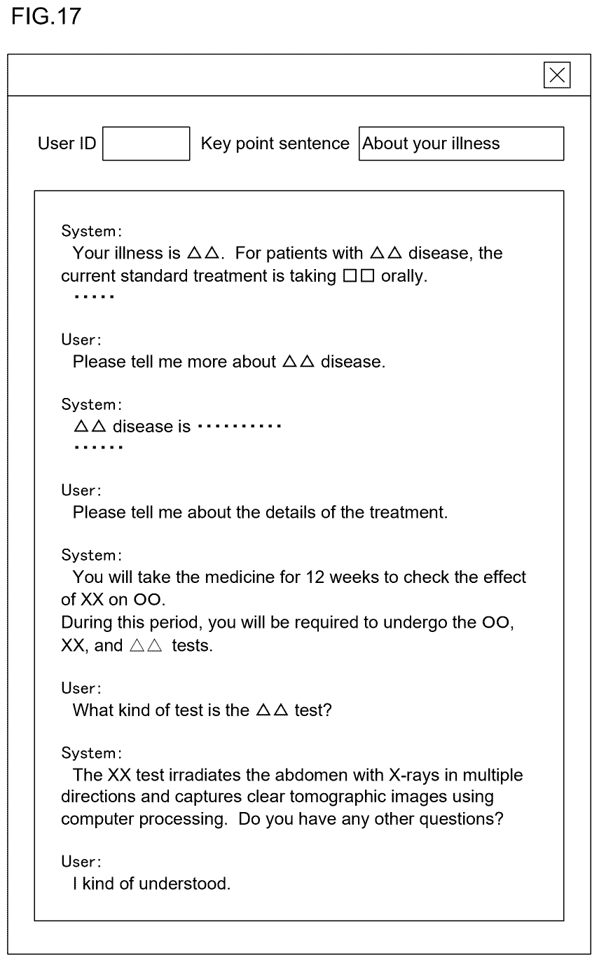

A dialogue log shown in FIG. 17, which will be described below, can be displayed by operating the “details” icons displayed in the classifications of “difficult to understand” and “concerns”.

FIG. 17 is a view showing an example of a dialogue log between the user and the large language model 120. The dialogue log shown in FIG. 17 is displayed by operating the “details” icon displayed in “difficult to understand” which is the classification of the level of understanding corresponding to the key point sentence “About your illness” in FIG. 16. In FIG. 17, a “system” indicates the large language model 120.

As shown in FIG. 17, for example, when the system outputs explanatory sentences “Your illness is ΔΔ. For patients with ΔΔ disease, the current standard treatment is taking □□ orally . . . . ”, the user may ask a question “Please tell me more about ΔΔ disease.” When the system responds with “ΔΔ disease is . . . ”, the user asks a question “Please tell me about the details of the treatment.” When the system responds with “You will take the medicine for 12 weeks to check the effect of XX on ◯◯. During this period, you will be required to undergo the ◯◯, XX, and ΔΔ tests, the user asks a question “What kind of test is the XX test?”. When the system responds with “The XX test irradiates the abdomen with X-rays in multiple directions and captures clear tomographic images using computer processing. Do you have any other questions?”, the user responds with “I kind of understood.”

The doctor can easily understand what points the user was unsure about when asking the question and what points are difficult for the user to understand, with reference to the dialogue log. Therefore, when acquiring the informed consent form from the user, the doctor can determine which parts of the informed consent form should be explained in detail.

Further, for any parts that are difficult for the user to understand, the reference document DB 110 can be updated and reinforced based on the user's questions and the answers from the large language model 120. Therefore, this can be used to improve the explanations of other users. In addition, the user's concerns can be reflected in, for example, the electronic medical records of the user.

For example, when consent is acquired frequently, when the informed consent form is long (for example, 50 pages), or when there are many explanation items, or when the content of the explanation is difficult for the subject to understand at the site where consent is acquired, the time and effort required for the medical professionals to explain things to the subjects often depend on the explanation skills of the medical professionals and the level of understanding of the subjects. There are limitations to the time available to the medical professionals and the subjects, or the workload and time of the medical professionals that can be allocated to explaining the informed consent form and to acquiring consent. For this reason, a case is assumed where it is difficult to achieve the acquisition of consent from the subjects after the subjects fully understand the content of the informed consent form, which is the original purpose of the informed consent form, at the limited time and workload of both the subjects and the medical professionals, which is a burden on both the medical professionals and the subjects.

However, according to this embodiment, the large language model 120 is trained using information, such as the informed consent form 111, the FAQ 112, and the scientific background explanatory document 113, and the know-how accumulated by the experienced medical professionals. The large language model 120 explains the content of the informed consent form to the subject through voice or sentences, and the subject has a dialogue with the large language model 120 about the content of the informed consent form through questions and requests such that the subject can understand the content of the informed consent form. The system can provide support to acquire consent with sufficient understanding. In addition, it is possible to significantly reduce the workload and burden on the medical professionals who explain the informed consent form or acquire consent.

Further, according to this embodiment, the information processing device 50 processes the dialogue (exchange of questions and answers thereto) between a user (a subject, a patient, or the like) and the large language model 120 to generate a dialogue log. In addition, for the informed consent form, the points that the user understood, the points that the user does not understand, the points that are difficult for the user to understand, or the points that the user is worried or concerned about can be collected as the consent status, and the collected consent status can be provided to the medical professionals such as the doctors. The medical professionals, such as the doctors, can explain the informed consent form to the user based on the provided consent status. Therefore, for example, it is not necessary to explain again the points that the user has already understood, which makes it possible to reduce the burden on the medical professionals such as the doctors, in acquiring consent. In addition, for the points that the user does not understand, the points that are difficult for the user to understand, or the points that the user is worried or concerned about, the medical professionals, such as the doctors, can explain the points again to prevent consent from being acquired in a state in which the user does not fully understand the informed consent form.

Further, according to this embodiment, the points that the user does not understand, the points that are difficult for the user to understand, or the points that the user is worried or concerned about are collected as the consent status, and the collected consent status is used as a new reference document for the large language model 120, which makes it possible to improve the accuracy and appropriateness of the answers by the large language model 120. This can be useful for improving the informed consent forms for new users in the future.

This embodiment is not limited to the explanation of the informed consent form and the acquisition of consent in clinical research and clinical trials, but can also be applied to cases where some medical procedure or surgery is performed on patients or the like.

(Supplementary Note 1) An information processing program causes a computer to execute: acquiring a key point sentence related to content of a consent form for a medical procedure or a clinical trial; reading a sentence related to the acquired key point sentence from a database; acquiring an explanatory sentence generated by inputting the key point sentence and the read sentence to a language model; and outputting the acquired explanatory sentence.

(Supplementary Note 2) The information processing program according to Supplementary Note 1 causes the computer to further execute reading a sentence related to the key point sentence from the database based on features calculated from the key point sentence and features associated with the sentence.

(Supplementary Note 3) The information processing program according to Supplementary Note 1 or 2 causes the computer to further execute inputting a language and knowledge level of an explainee to the language model.

(Supplementary Note 4) The information processing program according to any one of Supplementary Notes 1 to 3 causes the computer to further execute: preparing a plurality of the key point sentences in association with a presentation order; acquiring the key point sentence in the presentation order; and outputting the explanatory sentence corresponding to the acquired key point sentence.

(Supplementary Note 5) The information processing program according to Supplementary Note 4 causes the computer to further execute: outputting a question sentence asking for understanding together with the explanatory sentence; acquiring an answer to the question sentence; and when it is determined that the explainee has understood based on the acquired answer, outputting the explanatory sentence corresponding to the next key point sentence.

(Supplementary Note 6) The information processing program according to Supplementary Note 5 causes the computer to further execute: when it is determined that the explanee has not understood and the question sentence has been acquired based on the acquired answer, reading the sentence related to the question sentence from the database; acquiring an answer sentence generated by inputting the question sentence and the read sentence to the language model; and outputting the acquired answer sentence.

(Supplementary Note 7) The information processing program according to Supplementary Note 6 causes the computer to further execute reading the sentence related to the question sentence from the database based on features calculated from the question sentence and features associated with the sentence.

(Supplementary Note 8) The information processing program according to Supplementary Note 7 causes the computer to further execute: preparing a plurality of the key point sentences in association with a presentation order; outputting a question sentence asking for understanding together with the answer sentence; acquiring an answer to the question sentence; and, when it is determined that the explainee has understood based on the acquired answer, outputting the explanatory sentence corresponding to the next key point sentence in the presentation order.

(Supplementary Note 9) The information processing program according to any one of Supplementary Notes 6 to 8 causes the computer to further execute, when the question sentence is unethical or inconsistent with the facts, outputting a sentence pointing out that the question sentence is unethical or inconsistent with the facts and a sentence explaining that the question sentence is not capable of being answered.

(Supplementary Note 10) The information processing program according to any one of Supplementary Notes 1 to 9 causes the computer to further execute giving, to the language model, an instruction indicating that the explanatory sentence does not contain false information, and unethical and harmful content.

(Supplementary Note 11) The information processing program according to any one of Supplementary Notes 6 to 8 causes the computer to further execute giving, to the language model, an instruction indicating that the answer sentence does not contain false information, and unethical and harmful content.

(Supplementary Note 12) An information processing method executed by a computer includes: acquiring a key point sentence related to content of a consent form for a medical procedure or a clinical trial; reading a sentence related to the acquired key point sentence from a database; acquiring an explanatory sentence generated by inputting the key point sentence and the read sentence to a language model; and outputting the acquired explanatory sentence.

(Supplementary Note 13) An information processing device includes a control unit, and the control unit acquires a key point sentence related to content of a consent form for a medical procedure or a clinical trial, reads a sentence related to the acquired key point sentence from a database, acquires an explanatory sentence generated by inputting the key point sentence and the read sentence to a language model, and outputting the acquired explanatory sentence.

The matters described in each embodiment can be combined with each other. In addition, the independent and dependent claims described in the claims can be combined with each other in any and all combinations, regardless of the form of reference. Further, the claims may be described in a format in which a claim refers to two or more other claims (multiple claim format), but the format of the claims is not limited thereto. The claims may be described in a format in which a multiple dependent claim (multi-multi claim) refers to at least one multiple dependent claim.

It is to be noted that, as used herein and in the appended claims, the singular forms “a”, “an”, and “the” include plural referents unless the context clearly dictates otherwise.

As this invention may be embodied in several forms without departing from the spirit of essential characteristics thereof, the present embodiments are therefore illustrative and not restrictive, since the scope of the invention is defined by the appended claims rather than by the description preceding them, and all changes that fall within metes and bounds of the claims, or equivalence of such metes and bounds thereof are therefore intended to be embraced by the claims.

What is claimed is:

1. A computer readable non-transitory recording medium recording an information processing program causing a computer to execute:
acquiring a key point sentence related to content of a consent form for a medical procedure or a clinical trial;
reading a sentence related to the acquired key point sentence from a database;
acquiring an explanatory sentence generated by inputting the key point sentence and the read sentence to a language model; and
outputting the acquired explanatory sentence.

2. The computer readable non-transitory recording medium recording the information processing program according to claim 1, causing the computer to further execute:
reading a sentence related to the key point sentence from the database based on features calculated from the key point sentence and features associated with the sentence.

3. The computer readable non-transitory recording medium recording the information processing program according to claim 1, causing the computer to further execute:
inputting a language and knowledge level of an explainee to the language model.

4. The computer readable non-transitory recording medium recording the information processing program according to claim 1, causing the computer to further execute:
preparing a plurality of the key point sentences in association with a presentation order;
acquiring the key point sentence in the presentation order; and
outputting the explanatory sentence corresponding to the acquired key point sentence.

5. The computer readable non-transitory recording medium recording the information processing program according to claim 4, causing the computer to further execute:
outputting a question sentence asking for understanding together with the explanatory sentence;
acquiring an answer to the question sentence; and
when it is determined that an explainee has understood based on the acquired answer, outputting the explanatory sentence corresponding to the next key point sentence.

6. The computer readable non-transitory recording medium recording the information processing program according to claim 5, causing the computer to further execute:
when it is determined that the explainee has not understood and the question sentence has been acquired based on the acquired answer, reading the sentence related to the question sentence from the database;
acquiring an answer sentence generated by inputting the question sentence and the read sentence to the language model; and
outputting the acquired answer sentence.

7. The computer readable non-transitory recording medium recording the information processing program according to claim 6, causing the computer to further execute:
reading the sentence related to the question sentence from the database based on features calculated from the question sentence and features associated with the sentence.

8. The computer readable non-transitory recording medium recording the information processing program according to claim 7, causing the computer to further execute:
preparing a plurality of the key point sentences in association with a presentation order;
outputting a question sentence asking for understanding together with the answer sentence;
acquiring an answer to the question sentence; and
when it is determined that the explainee has understood based on the acquired answer, outputting the explanatory sentence corresponding to the next key point sentence in the presentation order.

9. The computer readable non-transitory recording medium recording the information processing program according to claim 6, causing the computer to further execute:
when the question sentence is unethical or inconsistent with the facts, outputting a sentence pointing out that the question sentence is unethical or inconsistent with the facts and a sentence explaining that the question sentence is not capable of being answered.

10. The computer readable non-transitory recording medium recording the information processing program according to claim 1, causing the computer to further execute:
giving, to the language model, an instruction indicating that the explanatory sentence does not contain false information, and unethical and harmful content.

11. The computer readable non-transitory recording medium recording the information processing program according to claim 6, causing the computer to further execute:
giving, to the language model, an instruction indicating that the answer sentence does not contain false information, and unethical and harmful content.

12. An information processing method executed by a computer, the information processing method comprising:
acquiring a key point sentence related to content of a consent form for a medical procedure or a clinical trial;
reading a sentence related to the acquired key point sentence from a database;
acquiring an explanatory sentence generated by inputting the key point sentence and the read sentence to a language model; and
outputting the acquired explanatory sentence.

13. An information processing device comprising:
a control unit,
wherein the control unit
acquires a key point sentence related to content of a consent form for a medical procedure or a clinical trial,
reads a sentence related to the acquired key point sentence from a database,
acquires an explanatory sentence generated by inputting the key point sentence and the read sentence to a language model, and
outputting the acquired explanatory sentence.
